(19) United States
(12) Patent Application Publication
(10) Pub. No.: US 2023/0372996 A1
(43) Pub. Date: Nov. 23, 2023

(54) INSPECTION CUP FOR INSPECTING IMPURITY IN MOLTEN METAL FOR DIE CASTING AND METHOD OF INSPECTING IMPURITY IN MOLTEN METAL FOR DIE CASTING USING INSPECTION CUP

(71) Applicants: HYUNDAI MOTOR COMPANY, Seoul (KR); KIA CORPORATION, Seoul (KR)

(72) Inventors: Jeongtae Maeng, Ulsan (KR); Chung Hwan Kim, Ulsan (KR)

(21) Appl. No.: 18/230,333

(22) Filed: Aug. 4, 2023

Related U.S. Application Data
(62) Division of application No. 17/524,178, filed on Nov. 11, 2021.

(30) Foreign Application Priority Data
May 11, 2021 (KR) 10-2021-0060441

(51) Int. Cl. B22C 9/08; B22D 41/01

(52) U.S. Cl. CPC B22C 9/086; B22D 41/01

(57) ABSTRACT

Disclosed is an inspection cup for inspecting an impurity in molten metal for die casting. The inspection cup for inspecting an impurity in molten metal for die casting according to an exemplary embodiment of the present disclosure is configured to detect an impurity contained in the molten aluminum and includes a cup body having a molten metal accommodation portion capable of accommodating a predetermined amount of molten aluminum, and the molten metal accommodation portion is shaped such that a cross-sectional area thereof gradually decreases from an upper end toward a lower end.

### CROSS REFERENCE TO RELATED APPLICATIONS

The present application is a divisional of U.S. application Ser. No. 17/524,178 filed Nov. 11, 2021, which claims priority to Korean Patent Application No. 10-2021-0060441 filed in the Korean Intellectual Property Office on May 11, 2021. The entire disclosure contents of these applications are herewith incorporated by reference into the present application.

### BACKGROUND

### (a) Field

The present disclosure relates to an apparatus for inspecting an impurity in molten metal for die casting, and more particularly, to an inspection cup for inspecting an impurity in molten metal for die casting, the inspection cup being configured to inspect an impurity contained in molten aluminum, and a method of inspecting an impurity in the molten metal using the inspection cup.

### (b) Description of the Related Art

In general, die casting refers to a process method of manufacturing a component having a predetermined shape by injecting a melted material (hereinafter, referred to as ‘molten metal’) into a die.

For example, components for a vehicle engine are manufactured by die casting using molten aluminum. The quality of the casting components made of aluminum may depend on impurities such as oxides contained in the molten aluminum.

As an example of a method of inspecting an impurity contained in the molten aluminum, a specimen made by coagulating the molten aluminum is cut and an impurity existing on a cut surface is detected with the naked eye.

However, the inspection method cannot detect a micrometer-sized fine impurity (e.g., a fine oxide at a level of 50 μm) that cannot be detected with the naked eye. The fine impurity may degrade castability of the molten aluminum and cause a defect of the casting component.

The above information disclosed in this Background section is only for enhancement of understanding of the background of the disclosure and therefore it may contain information that does not form the prior art that is already known in this country to a person of ordinary skill in the art.

### SUMMARY

The present disclosure has been made in an effort to provide an inspection cup for inspecting an impurity in molten metal for die casting, the inspection cup being configured to easily inspect a fine impurity contained in molten aluminum, and a method of inspecting an impurity in the molten metal using the inspection cup.

An exemplary embodiment of the present disclosure provides an inspection cup for inspecting an impurity in molten metal for die casting. The inspection cup is configured to detect an impurity contained in molten aluminum. The inspection cup may include a cup body having a molten metal accommodation portion configured to accommodate a predetermined amount of the molten aluminum, in which the molten metal accommodation portion is shaped such that a cross-sectional area thereof gradually decreases from an upper end toward a lower end.

In addition, the cup body may include an upper-end opening portion having an annular shape and connected to an upper end of the molten metal accommodation portion.

In addition, the molten metal accommodation portion may have a molten metal accommodation space having a circular cross-section in a transverse direction.

In addition, a thickness of the cup body between an outer peripheral surface and an inner peripheral surface may gradually increase from an upper end to a lower end.

In addition, the cup body may include a first extension portion extending in a radially outward direction from an edge of the upper end of the molten metal accommodation portion, and a second extension portion extending obliquely upward from a radially outer end of the first extension portion.

In addition, the cup body may further include a third extension portion extending in the radially outward direction from an upper end of the second extension portion.

In addition, the first extension portion, the second extension portion, and the third extension portion may form the upper-end opening portion.

In addition, the inner peripheral surface may be connected to an inner lower surface of the molten metal accommodation portion through a round portion.

In addition, an outer diameter of a lower end of the upper-end opening portion may be equal to a height of the molten metal accommodation portion.

In addition, a thickness of the upper-end opening portion may be equal to a thickness between an inner lower surface of the molten metal accommodation portion and a lower surface of the cup body.

In addition, the molten metal accommodation portion may include at least one rib protruding at least upward from an inner lower surface thereof.

In addition, the cup body may be made of steel or ceramic.

Another embodiment of the present disclosure provides a method of inspecting an impurity in molten metal for die casting which inspects the impurity contained in molten aluminum using the above-mentioned inspection cup. The method may include (a) preparing the cup body having the molten metal accommodation portion, (b) preheating the cup body to a preset temperature, (c) injecting a predetermined amount of the molten aluminum into the molten metal accommodation portion of the cup body, (d) coagulating the molten aluminum, (e) separating a specimen, made by coagulating the molten aluminum, from the cup body and cutting the specimen in a longitudinal direction, and (f) scanning a cut surface of the specimen with an inspection scanner and detecting, by a controller, an impurity index of the specimen.

In addition, the cup body may be made of steel or ceramic.

In addition, the preheating of the cup body may include injecting the molten aluminum, which is melted at a preset temperature, into the molten metal accommodation portion, and emptying the molten metal accommodation portion after a preset time.

In addition, the method may further include polishing the cut surface of the specimen after the cutting of the specimen.

According to the embodiment of the present disclosure, the impurity contained in the molten aluminum may be easily detected, thereby preventing a deterioration in castability (fluidity) of the molten aluminum caused by the impurity and reducing the occurrence of defect of the casting component caused by the impurity.

Other effects, which may be obtained or expected by the embodiments of the present disclosure, will be directly or implicitly disclosed in the detailed description on the embodiments of the present disclosure. That is, various effects expected according to the embodiments of the present disclosure will be disclosed in the detailed description to be described below.

### BRIEF DESCRIPTION OF THE FIGURES

Because the drawings are provided for reference to describe embodiments of the present disclosure, the technical spirit of the present disclosure should not be construed as being limited to the accompanying drawings.

FIG. 1 is a perspective view illustrating an inspection cup for inspecting an impurity in molten metal for die casting according to an embodiment of the present disclosure.

FIG. 2 is a top plan view illustrating the inspection cup for inspecting an impurity in molten metal for die casting according to the embodiment of the present disclosure.

FIG. 3 is a cross-sectional view illustrating the inspection cup for inspecting an impurity in molten metal for die casting according to the embodiment of the present disclosure.

FIG. 4 is a flowchart schematically illustrating a method of inspecting an impurity in molten metal for die casting according to the embodiment of the present disclosure.

FIGS. 5A, 5B, 6, 7, 8, 9, 10, 11, and 12 are views for explaining the method of inspecting an impurity in molten metal for die casting according to the embodiment of the present disclosure.

FIG. 13A is a cross-sectional view illustrating an inspection cup for inspecting an impurity in molten metal for die casting according to another embodiment of the present disclosure.

FIG. 13B is a perspective view of a specimen made from the inspection cup shown in FIG. 13A.

### DETAILED DESCRIPTION

Hereinafter, embodiments of the present disclosure will be described in detail with reference to the accompanying drawings so that those with ordinary skill in the art to which the present disclosure pertains may easily carry out the embodiments. However, the present disclosure may be implemented in various different ways and is not limited to the embodiments described herein.

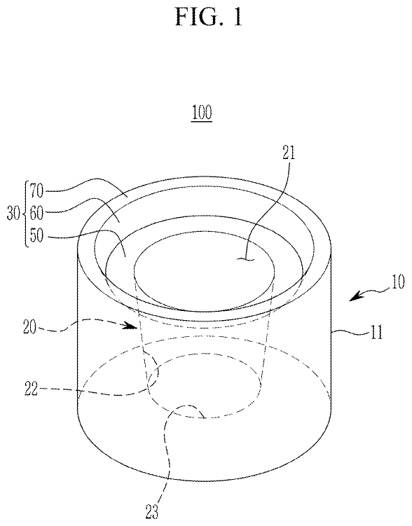

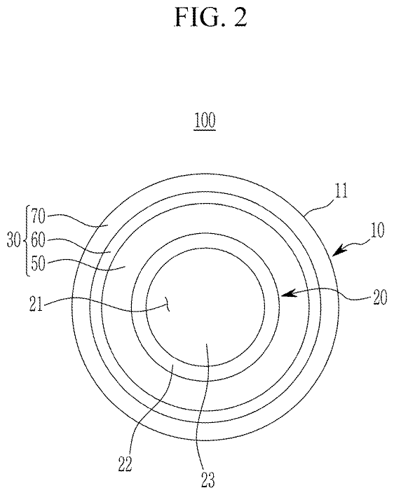

FIG. 1 is a perspective view illustrating an inspection cup for inspecting an impurity in molten metal for die casting according to an embodiment of the present disclosure, and FIG. 2 is a top plan view illustrating the inspection cup for inspecting an impurity in molten metal for die casting according to the embodiment of the present disclosure.

As illustrated in FIGS. 1 and 2, an inspection cup 100 for inspecting an impurity in molten metal for die casting according to an embodiment of the present disclosure may be applied to a high-pressure or low-pressure die casting process capable of manufacturing a product with a desired shape by injecting the molten metal made of aluminum, magnesium, or an alloy thereof into a die.

As one example, the die casting process may be a process of manufacturing an aluminum component having a desired shape, for example, a component for a vehicle body which is assembled with the vehicle body, by injecting molten aluminum into a die casting mold.

In the present specification, the terms ‘upper end portion,’ ‘upper portion’, ‘upper end’ or ‘upper surface’ of a component means an end portion, a portion, an end, or a surface of the component which is disposed at a relative upper side, and the terms ‘lower end portion,’ ‘lower portion’, ‘lower end’, or ‘lower surface’ of a component means an end portion, a portion, an end, or a surface of the component which is disposed at a relatively lower side.

In addition, in the present specification, an end (e.g., one end or the other end) of a component means an end of the component in any one direction, and an end portion (e.g., one end portion or the other end portion) of a component means a predetermined portion of the component that includes the end of the component.

The inspection cup 100 for inspecting an impurity in molten metal for die casting according to the embodiment of the present disclosure has a structure capable of inspecting a fine impurity (e.g., a fine oxide at a level of 50 μm) contained in molten aluminum 1.

To this end, the inspection cup 100 for inspecting an impurity in molten metal for die casting according to the embodiment of the present disclosure includes a cup body 10.

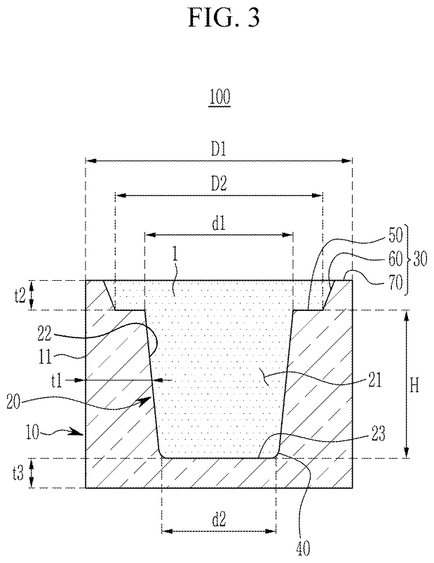

FIG. 3 is a cross-sectional view illustrating the inspection cup for inspecting an impurity in molten metal for die casting according to the embodiment of the present disclosure.

As illustrated in FIGS. 1 to 3, in the embodiment of the present disclosure, an upper end of the cup body 10 is opened, and a lower end of the cup body 10 is closed.

The cup body 10 may be made of various materials. As one example, the cup body 10 may be made of carburized and heat-treated steel. As another example, the cup body 10 may be made of ceramic.

The cup body 10 may include a molten metal accommodation portion 20, an upper-end opening portion 30, a first extension portion 50, a second extension portion 60, and a third extension portion 70.

The molten metal accommodation portion 20 is formed between an upper end and a lower end of the cup body 10. The molten metal accommodation portion 20 is configured to accommodate a predetermined amount of the molten aluminum 1.

The molten metal accommodation portion 20 may be shaped such that a cross-sectional area thereof gradually decreases from the upper end toward the lower end of the cup body 10. In one example, the molten metal accommodation portion 20 has a molten metal accommodation space 21 having a circular cross-section in a transverse direction. A diameter of the molten metal accommodation space 21 may gradually decrease from the upper end toward the lower end.

In one example, the upper-end opening portion 30 has an annular shape and is connected to an upper end of the molten metal accommodation portion 20.

In one example, the cup body 10 has an outer peripheral surface 11 having a constant diameter. Further, since the molten metal accommodation portion 20 has the circular cross-section in the transverse direction, the cup body 10 includes an inner peripheral surface 22 corresponding to the outer peripheral surface of the molten metal accommodation portion 20.

Since the molten metal accommodation portion 20 is shaped such that the cross-sectional area thereof gradually decreases from the upper end toward the lower end of the cup body 10, a thickness t1 of the cup body 10, between the outer peripheral surface 11 and the inner peripheral surface 22, gradually increases from the upper end toward the lower end.

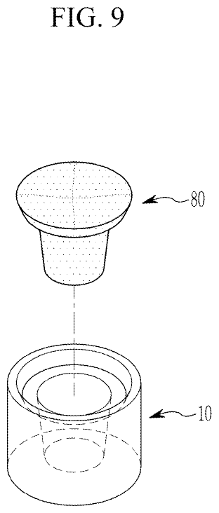

Further, the cup body 10 includes an inner lower surface 23 corresponding to a lower surface of the molten metal accommodation portion 20. The inner lower surface 23 is connected to the inner peripheral surface 22 through a round portion 40 having a predetermined radius. Therefore, a specimen 80 (see FIG. 9) made by coagulating the molten metal may be easily separated from the cup body 10.

The first extension portion 50 extends in a radially outward direction from an edge of the upper end of the molten metal accommodation portion 20. The radially outward direction may be defined as a direction from the edge of the upper end of the molten metal accommodation portion 20 toward the outer peripheral surface 11 of the cup body 10.

The second extension portion 60 extends obliquely upward from a radially outer end of the first extension portion 50. For example, a diameter of the second extension portion 60 may gradually increase upward.

Further, the third extension portion 70 extends in the radially outward direction from an upper end of the second extension portion 60.

The first extension portion 50, the second extension portion 60, and the third extension portion 70 may formed as the upper-end opening portion 30 as mentioned above.

Meanwhile, the inspection cup 100 for inspecting an impurity in molten metal for die casting according to the embodiment of the present disclosure may have various dimensions. Hereinafter, specifications of the inspection cup 100 according to one example will be described below in detail. However, the embodiment of the present disclosure is not limited to the exemplary specifications.

In the embodiment of the present disclosure, it is assumed that an outer diameter of the cup body 10 is D1, an outer diameter of the first extension portion 50 (corresponding to an outer diameter of a lower end of the upper-end opening portion) is D2, an inner diameter of the first extension portion 50 (corresponding to an outer diameter of an upper end of the molten metal accommodation portion 20) is d1, and an outer diameter of the lower end of the molten metal accommodation portion 20 is d2.

In one example, the outer diameter D1 of the cup body 10 may be 35 to 45 mm, the outer diameter D2 of the first extension portion 50 may be 25 to 35 mm, the inner diameter d1 of the first extension portion 50 may be 15 to 25 mm, and the outer diameter d2 of the lower end of the molten metal accommodation portion 20 may be 10 to 20 mm.

Therefore, according to the specifications of the inspection cup 100, D1−D2=10 mm, D1−d1=20 mm, D1−d2=25 mm, D2−d1=10 mm, D2−d2=15 mm, and d1−d2=5 mm.

Further, the thickness t1 of the cup body 10 between the outer peripheral surface 11 and the inner peripheral surface 22 may be 10 to 12.5 mm at any position in upward/downward direction.

In addition, a thickness t2 of the upper-end opening portion 30 may be equal to a thickness t3 between the inner lower surface 23 and the lower surface of the cup body 10. In one example, the thicknesses t2 and t3 may each be 5 mm.

Further, the outer diameter D2 of the first extension portion 50 may be equal to a height H of the molten metal accommodation portion 20. Further, the molten metal accommodation portion 20 may accommodate a predetermined amount (e.g., 10 to 30 cc) of the molten aluminum.

The above-mentioned ranges may be determined to be most suitable for the impurity inspection for the molten aluminum 1 based on a coagulation speed of the molten aluminum 1 that varies depending on various specifications of the inspection cup 100.

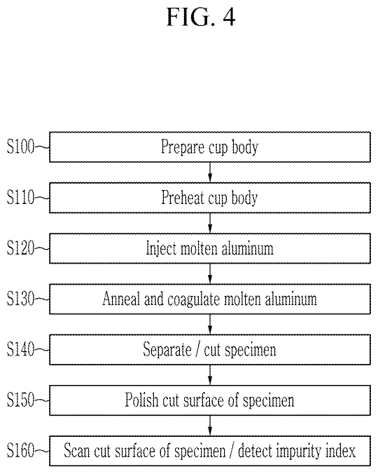

Hereinafter, a method of inspecting an impurity in molten metal for die casting using the inspection cup 100 for inspecting an impurity in molten metal for die casting according to the embodiment of the present disclosure configured as described above will be described in detail with reference to FIGS. 4 to 12.

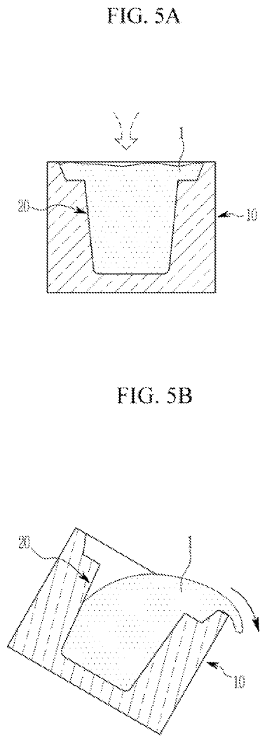

FIG. 4 is a flowchart schematically illustrating a method of inspecting an impurity in molten metal for die casting according to the embodiment of the present disclosure, and FIGS. 5 to 12 are views for explaining the method of inspecting an impurity in molten metal for die casting according to the embodiment of the present disclosure.

As illustrated in FIG. 4, the method of inspecting an impurity in molten metal for die casting according to the embodiment of the present disclosure starts with step S100 and sequentially performs step S110, step S120, step S130, step S140, and step S160. In addition, step S150 may be optionally performed between step S140 and the step S160.

As illustrated in FIG. 3, in the step S100, the cup body 10 having the molten metal accommodation portion 20 is prepared. As described above, the cup body 10 is manufactured in advance using steel or ceramic. The cup body 10 has the molten metal accommodation portion 20 shaped such that the cross-sectional area thereof gradually decreases from the upper end toward the lower end. The thickness of the cup body 10 between the outer peripheral surface 11 and the inner peripheral surface 22 gradually increases from the upper end toward the lower end.

In the step S100, the upper-end opening portion 30 having an annular shape is formed at the upper end of the cup body 10 and connected to the molten metal accommodation portion 20. Further, the upper-end opening portion 30 may include the first extension portion 50, the second extension portion 60, and the third extension portion 70.

As illustrated in FIG. 5, in the step S110, the cup body 10 is preheated to a preset temperature. The cup body 10 may be preheated by the molten aluminum 1. In one example, the molten aluminum 1, which is melted at a preset temperature (e.g., about 700° C.), is accommodated for a predetermined time (e.g., 30 seconds or more) in the molten metal accommodation portion 20 of the cup body 10. Therefore, the cup body 10 may be preheated to a predetermined temperature (e.g., about 200 to 300° C.) by heat of the molten aluminum 1.

That is, as illustrated in FIG. 5A, in the step S110, the molten aluminum 1 is accommodated in the molten metal accommodation portion 20. As illustrated in FIG. 5B, when the predetermined time has elapsed, the molten metal accommodation portion 20 is emptied, such that the cup body 10 may be preheated.

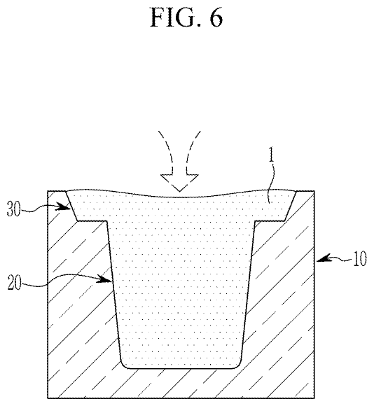

As illustrated in FIG. 6, in the step S120, the molten aluminum 1 is injected into the molten metal accommodation portion 20 of the preheated cup body 10. In one example, approximately 10 to 30 cc of the molten aluminum 1 may be accommodated in the molten metal accommodation portion 20 to the upper-end opening portion 30.

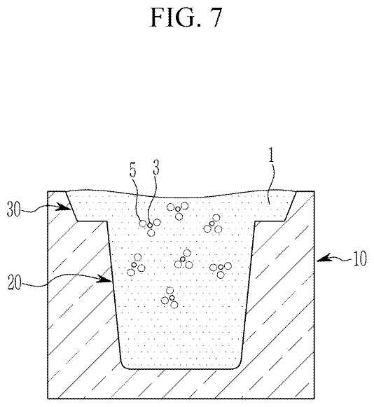

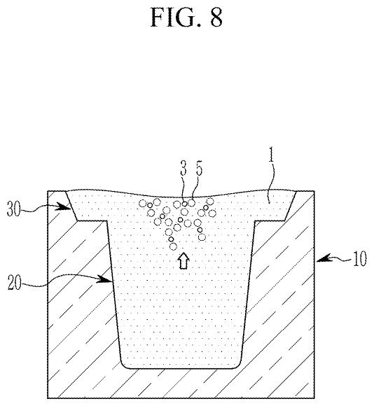

As illustrated in FIGS. 7 and 8, in the step S130, annealing is performed on the molten aluminum 1 for a predetermined time (e.g., 7 minutes or more) and the molten aluminum 1 is coagulated.

In the step S130, during the process in which the molten aluminum 1 is coagulated, micropores 5 are collected at the periphery of the impurities 3 such as oxides contained in the molten aluminum 1, as illustrated in FIG. 7. This situation may occur because surface energy of the impurity 3 is high and the molten metal accommodation portion 20 is shaped such that the cross-sectional area thereof gradually decreases from the upper end toward the lower end of the cup body 10. Therefore, the micropores 5 are collected at the periphery of the impurities 3 to decrease surface energy of the impurities 3.

As illustrated in FIG. 8, in the step S130, the impurities 3 float in the molten metal accommodation portion 20 toward the upper-end opening portion 30 in the state in which the micropores 5 are concentrated at the periphery of the impurities 3. This situation may occur because the molten metal accommodation portion 20 is shaped such that the cross-sectional area thereof gradually decreases from the upper end toward the lower end of the cup body 10. Further, the impurities 3 have higher density than aluminum, but the overall density of the combination of the impurities 3 and the micropores 5 becomes lower than the density of aluminum as the impurities 3 are combined with the micropores 5. Therefore, the impurities 3 may float because of a difference in density.

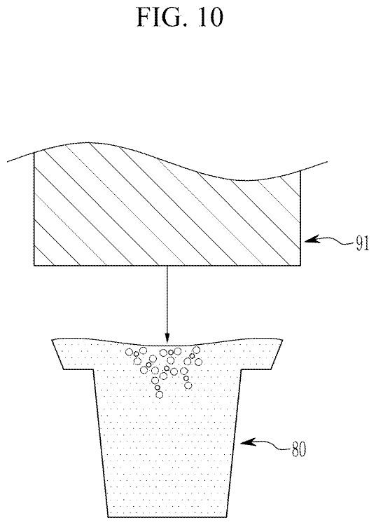

As illustrated in FIG. 9, in the step S140, the specimen 80 made by coagulating the molten aluminum is separated from the cup body 10. As illustrated in FIG. 10, the specimen 80 is cut in a longitudinal direction (upward/downward direction in the drawings) by a cutter 91 in various ways well known to those skilled in the art.

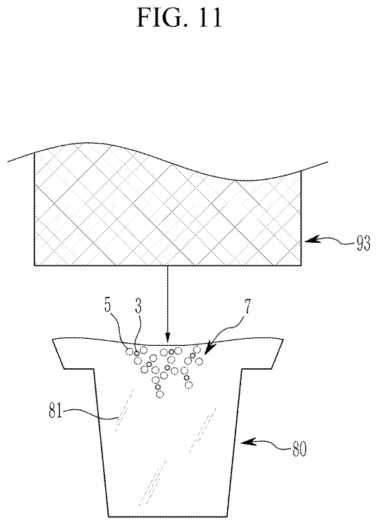

As illustrated in FIG. 11, in the optional step S150, a cut surface 81 of the specimen 80 is polished by a polisher 93 such as a belt grinder or sandpaper in various ways well known to those skilled in the art. In one example, the cut surface 81 of the specimen 80 may be polished to 200 to 1,000 meshes by the polisher 93. Therefore, the impurities 3 floated by the micropores 5 may be detected on the cut surface 81 of the specimen 80 with the naked eye. Hereinafter, a region in which the micropores 5 are concentrated at the periphery of the impurities 3 on the cut surface 81 of the specimen 80 is referred to as impurity pores 7.

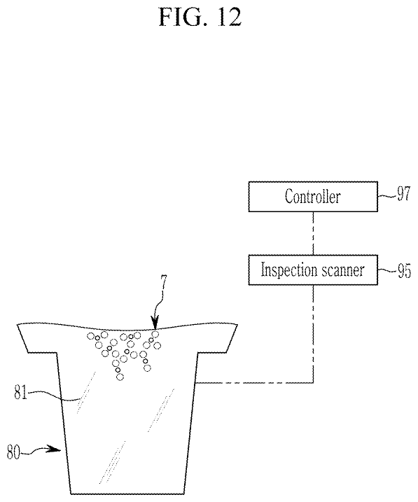

As illustrated in FIG. 12, in the step S160, an inspection scanner 95 scans the cut surface 81 of the specimen 80, and a controller 97 receives a signal corresponding to a scanning result and detects an impurity index of the specimen 80. In one example, the inspection scanner 95 may include a line laser scanner or a vision sensor well known to those skilled in the art.

In the step S160, the inspection scanner 95 extracts image data related to the cut surface 81 of the specimen 80 and transmits the image data to the controller 97. The controller 97 detects the impurity index of the specimen 80 using the image data received from the inspection scanner 95. To this end, the controller 97 may include one or more processors executed by a preset program. In particular, the controller 97 may include one or more processors that implement an image recognition function well known to those skilled in the art.

In one example, the controller 97 may generate an image based on the received image data and automatically recognize, from the image, the impurity pores 7 and the remaining region except for the impurity pores 7.

Therefore, the controller 97 may digitize a ratio of an area of the impurity pores 7 to an area of the entire cut surface 81 of the specimen 80 and output the numerical value as the impurity index. In this case, the impurity content may decrease as the numerical value of the impurity index decreases, and the impurity content may increase as the numerical value increases.

In another example, the controller 97 may transmit the impurity index or an inspection result according to the impurity index to a display (not illustrated), and the display may visually output the impurity index or the inspection result.

As described above, the inspection cup 100 for inspecting an impurity in molten metal for die casting according to the embodiment of the present disclosure and the method of inspecting an impurity in molten metal for die casting using the inspection cup 100 may inspect the impurities contained in the molten aluminum 1.

According to the embodiment of the present disclosure, the simple method using the inspection cup 100 may easily detect the impurity contained in the molten aluminum 1, thereby preventing a deterioration in castability (fluidity) of the molten aluminum caused by the impurity and reducing the occurrence of defect of the casting component caused by the impurity.

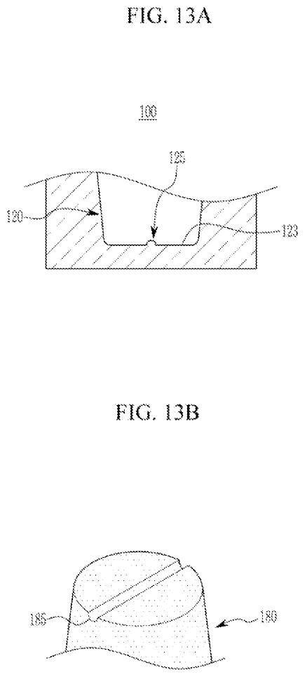

FIG. 13A is a cross-sectional view illustrating an inspection cup for inspecting an impurity in molten metal for die casting according to another embodiment of the present disclosure.

Referring to FIG. 13A, an inspection cup 200 for inspecting an impurity in molten metal for die casting according to another embodiment of the present disclosure may further include at least one rib 125 protruding upward at least from an inner lower surface 123 of a molten metal accommodation portion 120.

In the embodiment of the present disclosure, the rib 125 may be disposed on the inner lower surface 123 in the diameter direction. The rib 125 forms a cutting groove 185 in a corresponding lower surface of a specimen 180 coagulated in the molten metal accommodation portion 120. The specimen 180 may be easily cut by the cutting groove 185 formed by the rib 125, as shown in FIG. 13B.

Because the remaining configuration and operational effect of the inspection cup 200 for inspecting an impurity in molten metal for die casting according to another embodiment of the present disclosure are identical to those in the above-mentioned embodiment, a detailed description thereof will be omitted.

While this disclosure has been described in connection with what is presently considered to be practical exemplary embodiments, it is to be understood that the disclosure is not limited to the disclosed embodiments. On the contrary, it is intended to cover various modifications and equivalent arrangements included within the spirit and scope of the appended claims.

1. A method of inspecting an impurity in molten metal for die casting which inspects the impurity contained in molten aluminum using an inspection cup comprising a cup body having a molten metal accommodation portion configured to accommodate a predetermined amount of the molten aluminum, and wherein a cross-sectional area of the molten metal accommodation portion gradually decreases from an upper end toward a lower end, the method comprising:
preparing the cup body having the molten metal accommodation portion;
preheating the cup body to a preset temperature;
injecting a predetermined amount of the molten aluminum into the molten metal accommodation portion of the cup body;
coagulating the molten aluminum;
separating a specimen, made by coagulating the molten aluminum, from the cup body, and cutting the specimen in a longitudinal direction; and
scanning a cut surface of the specimen with an inspection scanner and detecting, by a controller, an impurity index of the specimen.

2. The method of claim 1, wherein the cup body is made of steel or ceramic.

3. The method of claim 1, wherein preheating the cup body comprises:
injecting the molten aluminum, which is melted at a preset temperature, into the molten metal accommodation portion; and
emptying the molten metal accommodation portion after a preset time.

4. The method of claim 1, further comprising polishing the cut surface of the specimen after cutting the specimen.
